(19) United States
(12) Patent Application Publication
(10) Pub. No.: US 2018/0161090 A1
(43) Pub. Date: Jun. 14, 2018

(54) NAVIGATED BIPOLAR FORCEPS

(71) Applicants: Amandeep Singh Pabla, Sacramento, CA (US); Jatinder Paul Singh, Elk Grove, CA (US)

(72) Inventors: Amandeep Singh Pabla, Sacramento, CA (US); Jatinder Paul Singh, Elk Grove, CA (US)

(21) Appl. No.: 15/839,561

(22) Filed: Dec. 12, 2017

Related U.S. Application Data
(60) Provisional application No. 62/432,756, filed on Dec. 12, 2016.

(51) Int. Cl. A61B 18/14

(52) U.S. Cl. CPC A61B 18/1445; A61B 2018/00898

(57) ABSTRACT

An all-in-one, light-weight navigated bipolar forceps allow the surgeon to stay focused on and not have to take their eyes off the surgical site that (s)he is operating on, such as the brain. The navigated bipolar forceps would have a navigated frame that would be positioned at the superior end of the bipolar forceps instrument. Like conventional bipolar forceps, the navigated bipolar forceps of the present invention may be useful for various procedures, such as bipolar electro surgery. The navigated frame would be incorporated into the instrument to provide an all-in-one solution that requires fewer instruments in the surgical field and retains the surgeon's focus on the surgical site. The navigated frame of the forceps can be designed to wirelessly connect to a machine for the navigational purposes. Software can be provided that permits the navigated bipolar forceps to support the instrument on various hospital-owned navigation machines.

### CROSS-REFERENCE TO RELATED APPLICATIONS

This application claims the benefit of U.S. provisional patent application No. 62/432,756, filed Dec. 12, 2016, the contents of which are herein incorporated by reference.

### BACKGROUND OF THE INVENTION

### 1. Field of the Invention

One or more embodiments of the invention relates generally to surgical tools. More particularly, the invention relates to navigated bipolar forceps that combines a navigated probe with a bipolar forceps.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Currently, a surgeon has to alternate between a navigated probe and a bipolar forceps. In doing so, the surgeon can lose focus on the surgical site. Moreover, current technology requires multiple instruments on the field.

In view of the foregoing, there is a need for improved bipolar forceps that include a navigated frame in a single instrument.

### SUMMARY OF THE INVENTION

Embodiments of the present invention provide navigated bipolar forceps comprising bipolar forceps; and a navigated frame attached as a superior end of the bipolar forceps, the navigated frame configured to wirelessly communicate with a navigation machine to determine a position of the bipolar forceps in space.

Embodiments of the present invention further provide a method for performing a surgery comprising connecting navigated bipolar forceps to a computer navigation machine, the navigated bipolar forceps including bipolar forceps and a navigated frame attached as a superior end of the bipolar forceps; and viewing a position of the bipolar forceps on a display based on a determined position of the bipolar forceps.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 is a top view of a navigated bipolar forceps according to an exemplary embodiment of the present invention;

FIG. 2 is a perspective view of the navigated bipolar forceps of FIG. 1;

FIG. 3 is a side view of the navigated bipolar forceps of FIG. 1;

FIG. 4 is a perspective end view of the navigated bipolar forceps of FIG. 1;

FIG. 5 is a schematic representation of the use of the navigated bipolar forceps of FIG. 1 in a surgical suite, for example, with a computer navigation system; and

FIG. 6 is an exploded perspective view of navigated bipolar forceps according to an exemplary embodiment of the present invention.

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention, and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide an all-in-one, light-weight navigated bipolar forceps that allow the surgeon to stay focused on and not have to take their eyes off the surgical site that (s)he is operating on, such as the brain. The navigated bipolar forceps would have a navigated frame that would be positioned at the superior (top, or distal) end of the bipolar forceps instrument. Like conventional bipolar forceps, the navigated bipolar forceps of the present invention may be useful for various procedures, such as bipolar electro surgery.

The device of the present invention would be light-weight and could be made from, for example, a carbon fiber material. The tips may be coated with a non-stick coating (such as Teflon®, for example). The size of the bipolar forceps would be similar to conventional, non-navigated forceps, and various tip sizes would be available, depending on the specific desired application. A navigated frame would be incorporated into the instrument to provide an all-in-one solution that requires fewer instruments in the surgical field and retains the surgeon's focus on the surgical site.

The navigated frame of the forceps of the present invention can be designed to wirelessly connect to a machine for the navigational purposes. Software can be provided that permits the navigated bipolar forceps to support the instrument on various hospital-owned navigation machines.

The navigated bipolar forceps of the present invention can improve patient safety and reduce operating room time by eliminating steps and maintaining the surgeon's focus on the surgical field.

The navigated bipolar forceps of the present invention may be useful in frameless stereotactic surgery of various body systems, such as brain or spinal surgery. Being frameless, the device of the present invention allows the surgeon to work under the scope without the frame interfering with his or her operation.

The navigated bipolar forceps of the present invention may include various additional features. These features may include, as non-limiting examples, irrigation and/or suction, lights, a cutting blade/scalpel, nerve stimulation capability, and the like.

The navigated bipolar forceps may have tips of various sizes, depending on the particular application. Typically, the tips may be from about 0.02 mm to about 2 mm. The tips may be straight, angled up or angled down, depending on application, and the angle may vary from a few degrees to up to 90 degrees or greater, for example. In some embodiments, the tips of the forceps may be formed from a material or be coated to be non-stick. In some embodiments, the tips of the forceps may be coated with chromium nitride coating.

The forceps may be operated with a cord or cordlessly. Moreover, the forceps may be formed as a disposable item or may be manufactured for cleaning, for example, autoclaving.

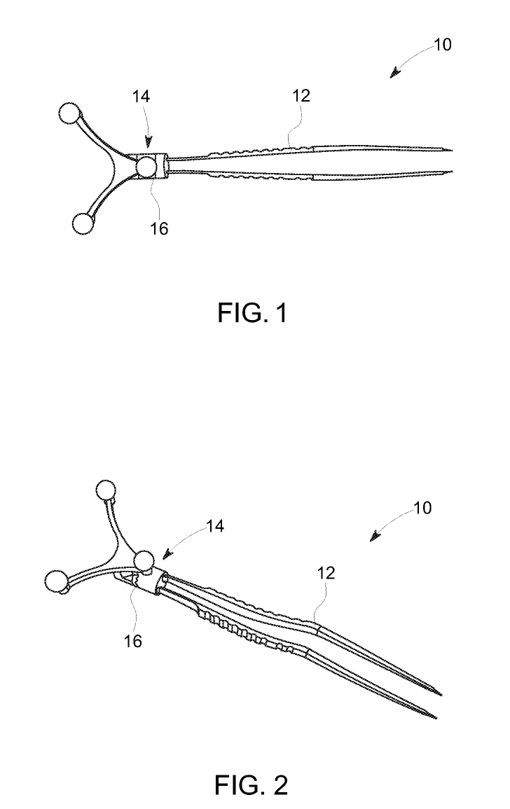

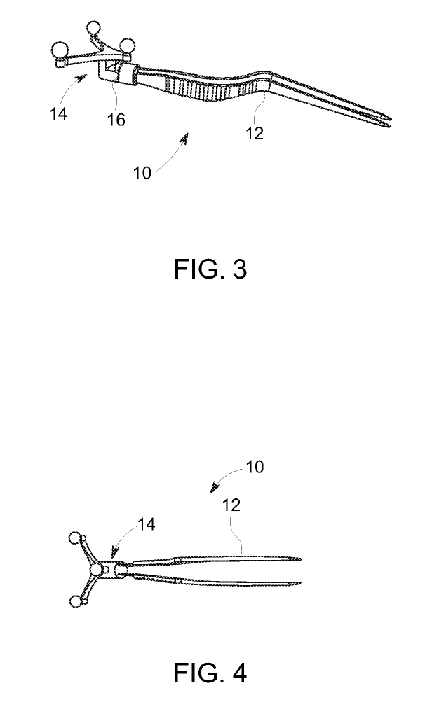

Referring to FIGS. 1 through 4, there is shown an exemplary embodiment of a navigated bipolar forceps 10 according to the present invention. The navigated bipolar forceps 10 can include bipolar forceps 12 and a navigated frame 14. A rotation joint 16 may interconnect the navigated frame 14 with the bipolar forceps 12. The rotation joint 16 may be useful to permit a user to better position the navigated bipolar forceps 10 during use thereof.

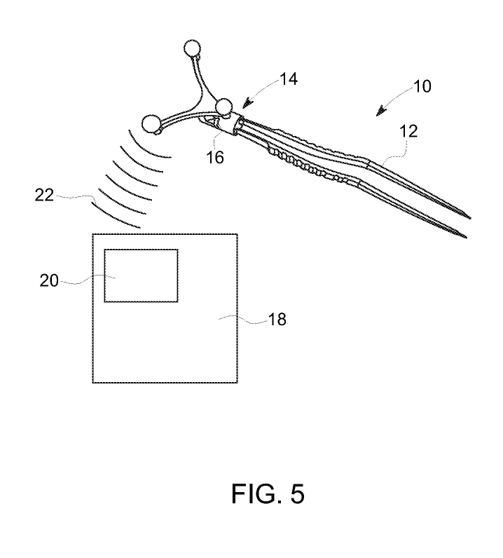

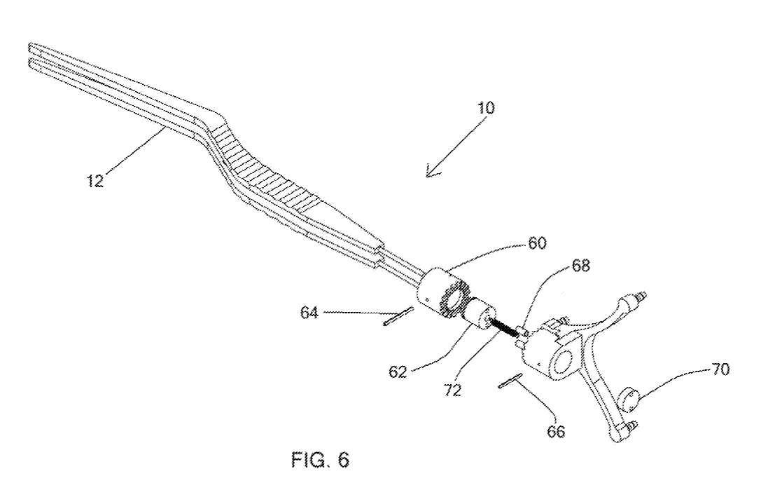

FIG. 5 shows a schematic representation of the navigated bipolar forceps 10 and a wireless connection 22 to a computer navigation machine 18 that may be present in, for example, a hospital surgical suite. The computer navigation machine 18 may include a display 20 for visualizing a position of the navigated bipolar forceps 10 in a predetermined location. While FIG. 5 shows the display 20 as part of the computer navigation machine 18, the display 20 may be any display as known in the art, including a heads-up display, a glasses display, or the like. FIG. 6 shows an exploded view of the navigated bipolar forceps 10, illustrating how a spring 72 can be disposed and held with pins 64, 66 to permit angle adjustment about adjusting piece 60. Additionally, insulators 62, 68, 70 can be seen that insulates the ends of the forceps 12 as they pass through the end of the navigated bipolar forceps 10.

All the features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. Navigated bipolar forceps comprising:
bipolar forceps; and
a navigated frame attached as a superior end of the bipolar forceps, the navigated frame configured to wirelessly communicate with a navigation machine to determine a position of the bipolar forceps in space.

2. The navigated bipolar forceps of claim 1, further comprising:
a rotation joint disposed between the bipolar forceps and the navigated frame, the rotation joint permitting a relative rotation between the bipolar forceps and the navigated frame.

3. The navigated bipolar forceps of claim 2, further comprising determining the relative rotation between the bipolar forceps and the navigated frame and sending the relative rotation data to the navigation machine.

4. A method for performing a surgery, comprising:
connecting navigated bipolar forceps to a computer navigation machine, the navigated bipolar forceps including bipolar forceps and a navigated frame attached as a superior end of the bipolar forceps;
viewing a position of the bipolar forceps on a display based on a determined position of the bipolar forceps.

5. The method of claim 4, wherein the navigated bipolar forceps connect wirelessly with the computer navigation machine.

6. The method of claim 4, further comprising permitting a relative rotation between the bipolar forceps and the navigated frame using a rotation joint disposed between the bipolar forceps and the navigated frame, the rotation joint.

7. The method of claim 6, further comprising determining the relative rotation between the bipolar forceps and the navigated frame and sending the relative rotation data to the computer navigation machine.
